(19) United States
(12) Patent Application Publication
(10) Pub. No.: US 2013/0319889 A1
(43) Pub. Date: Dec. 5, 2013

(54) SKIN CARE AND RESTORATION COMPOSITION AND METHODS

(71) Applicant: SURGEONS SKIN RECOVERY STICK, LLC, Hermosa Beach, CA (US)

(72) Inventor: Stephen A. DeSantis, Laguna Niguel, CA (US)

(21) Appl. No.: 13/905,072

(22) Filed: May 29, 2013

Related U.S. Application Data
(60) Provisional application No. 61/652,323, filed on May 29, 2012.

(51) Int. Cl. A61K 8/92; A61J 1/00

(52) U.S. Cl. CPC A61K 8/927; A61K 8/922; A61J 1/00
USPC 206/438; 424/537

(57) ABSTRACT

Topical compositions, such as lip balms and other products for lip care, include combinations of ingredients that condition and heal the lips and skin. The topical compositions may be configured to remain in place on the lips or skin for relatively long periods of time. Methods of making topical compositions are also disclosed.

### TECHNICAL FIELD

This disclosure relates generally to topical compositions, such as lip balms and other products for lip care. More specifically, this disclosure relates to topical compositions, including compositions for application to lips and other body parts, that include combinations of ingredients that condition and heal the lips and skin. In some embodiments, the topical compositions may be configured to remain in place on the lips or skin for relatively long periods of time.

### RELATED ART

Many skin care products and other topical compositions are commercially available. Many of the items of the line of skin care products marketed under the BERT'S BEES® brand include beeswax as a major component. BERT'S BEES® products are used primarily for chapped hands or lips. CHAPSTICK®, which is primarily intended for lip care, is another well-known topical composition. There are many similar products, some of which are based on or include beeswax, others of which are based on or include petroleum jelly and other petroleum-based products and other products are based on other materials.

Products designed for lip care are often very soft and easily rubbed off of an individual's lips.

### SUMMARY

A topical composition according to this disclosure may be configured to be used on any area of the body, including, but not limited to, the lips. The topical composition may be durable, or configured not to be easily rubbed off or removed. In some embodiments, the topical composition may be particularly useful for persons that are engaged in work that requires frequent hand washing and/or exposure to antiseptics or solvents. Some embodiments of topical compositions that incorporate teachings of this disclosure may be formulated for use by people with particularly sensitive or dry skin.

A topical composition, in various embodiments, may comprise a very durable long lasting protective skin product. The topical composition may include a variety of restoring and soothing oils with protective and healing properties in a convenient package.

In a specific, but non-limiting embodiment, a topical composition may include a combination of twenty (20) or more waxes and oils that provide healing features not found in other products, and various combinations of which may function synergistically. In some embodiments, the topical composition may be formulated to protect skin and/or lips, moisturize dry and/or chapped skin, facilitate the healing of injured lips or skin, heal injured lips or skin, or any combination of the foregoing. The topical composition may have antiseptic, antiviral, antifungal, anti-bacterial or anti-inflammatory properties, or any combination of these properties.

The topical composition may be configured for durability. In some embodiments, the topical composition may be configured to enable an individual to wash his or her hands without the need for re-application, to apply the topical composition before bed with the expectation that the topical composition will remain in place while the individual sleeps, etc.

Methods for making a topical composition may include adding components or ingredients in decreasing order of melting temperature. The temperature may decrease gradually over the course of mixing.

The topical composition may be carried by a container, or dispenser. Packages for the topical composition may include two or more containers, or dispensers. The different containers, or dispensers, of such a package may carry the same topical composition, but be labeled for different uses (e.g., one for use on lips, another for use on one or more other body parts, etc.). The containers, or dispensers, of such a package may, in some embodiments, be arranged in a piggy-back orientation (e.g., one on top of or next to the other, etc.).

Other aspects, as well as features and advantages of various aspects, of the disclosed subject matter will be apparent to those of ordinary skill in the art through consideration of the ensuing description and the appended claims.

### DETAILED DESCRIPTION

### 1. Composition of Embodiments of the Present Invention

Various embodiments of a topical composition include, consist essentially of or consist of the following ingredients, which may or may not be present in the amounts indicated:

| | |
|---|---|
| Beeswax | 1400 grams (g) |
| Lanolin | 400 g |
| Shea butter | 400 g |
| Cocoa butter | 400 g |
| Castor oil | 200 g |
| Jojoba oil | 200 g |
| Avocado oil | 100 g |
| Flax seed oil | 100 g |
| Grape seed oil | 50 g |
| Aloe vera oil | 50 g |
| Meadowform oil | 50 g |
| Hemp oil | 50 g |
| Sea buckthorn | 50 g |
| Baobab oil | 50 g |
| Carrot seed oil | 25 g |
| Arnica oil | 25 g |
| Vitamin E oil | 50,000 International Units (I.U.) |
| Vitamin D oil | 50.000 I.U. |
| Vitamin A oil | 50.000 I.U. |
| Tea tree oil | 15 g |
| Balsam fir oil | 5 g |
| Green tea oil | 10 g |

Referring to the foregoing list, the following is a more detail description of some of the ingredients:

| Bees Wax | C₁₅H₃₁COOC₃₀H₆₁ | |
|---|---|---|
| | Consists of Palmitate, Palmitoleate, | |
| | Hydroxypalmitate and Olepte Esters of Long | |
| | Chain Aliphatic Alcohols. | |
| | Melting Point | 62°-64° C. (144-147° F.) |
| Cocoa Butter | Complex Fat | |
| | Saturated Fat | 57-64% |
| | Stearic Acid | 24-37% |
| | Palmitic Acid | 22-29% |
| | Unsaturated Fat | 36-43% |
| | Mono Unsaturated Fat | 29-43% |
| | Oleic Acid | 29-41% |
| | Rich in Antioxidants, Prevents rancidity. | |
| | Shelf Life: 2-5 years | |
| | Melting Point | 34-38° C. |
| Shea Butter | Oleic Acid | 50-60% |
| | Palmitic Acid | ~-05% |
| | Linoleic Acid | ~-7.9% |
| | Stearic Acid | 35-45% |

| | |
|---|---|
| | Melting Point: 32-35° C. (89-95° F.) |
| Lanloin USP | Actually a wax. Not a fat. 97% Long chain |
| (ANHYDROUS) | waxy esters |
| | Melting Point 36-42° C. (97-108° F.) |
| Castor Oil | Nearly 90% of Fatty Acid content is Ricinoleic |
| | Acid (a Triglyceride of Fatty Acids) |
| | Antifungal properties, Anti-Inflammatory, |
| | Analgesic, Antibacterial |
| JoJoBa | Structurally closely resembles natural sebum. |
| | Actually a liquid Wax ester rather |
| | than an oil hypoallergenic, antimicrobial and |
| | Antifungal. Also, very stable with indefinite shelf life. |
| | Also acts as a preservative. |
| Avocado Oil | Rich in Vitamin A, D, & E. Helps increase the amount |
| | of Collagen in the skin. Also helps prevent itching. |
| Grape Seed Oil | Extremely light oil— Readily penetrates |
| | skin for deep restoration. |
| Aloe Vera Oil | Increases rate of skin repair, strength, antiviral, |
| | antifungal and antibacterial. Penetrating |
| | ability increases skins hydration and |
| | carries or transports other nutrients through |
| | the skin. Anti-Inflammatory. |
| Meadowfoam Oil | Rich in vitamin E and antioxidants. |
| | A very stable oil. Acts as a |
| | Preservative. Gives UV protection and skin |
| | the ability to retain \ Moisture. |
| Hempseed Oil | Anit-Inflammatory properties, rich in gamma |
| | linoleic acid and Omega-6, 3 & 9 oils. |
| | Restores damaged skin. |
| Carrot seed Oil | Expensive but has amazing effects on skin. Rich |
| | in Beta-Carotene Vitamins B, C, D & E. |
| | Also contains daucol, Alphe pinene, carotol |
| | Asorone, Limonene & Bisabolene. Stimulates cell |
| | growth, gives UV protection. Carotoid Daucol, |
| | Antifungal, Vasodilotory & Antioxidant. |
| Arnica Oil | Anti-Bacterial and Anti-Inflammatory |
| | properties. Improves wound healing. |
| | Also stimulates blood circulation. |
| Sea Buckthorn Oil | Rich in Carotenes, Tocopnerols, |
| | Phytosterols—All important for |
| Sea Buckthorn Oil | Healthy skin. High Anti-Oxidant |
| | nature. Aids in Healing due |
| | in part to Pro-Vitamin A Beta Carotene & |
| | Vitamin E. Anti-Bacterial—Inhibits |
| | growth of saphylocci and other Bacteria. |
| Tea Tree Oil | Antiseptic, Anti-Bacterial & Antifungal. |
| | Has strong activity Against staphylococcus |
| | aureus, including MRSA. (Methycillin |
| | Resistant Staph Aureus) |
| Balsam Fir Oil | Anitmicrobial, Analgesic, Antiseptic and Fragrant. |
| | Also Anti-Inflammatory. |
| Baobab Oil | Rich in vitamins A, E & D. Also rich in |
| | essential fatty acids, Omegas 3, 6 & 9, |
| | fatty acids. Extremely stable and very effective |
| | for treatment of Eczema & psoriasis. |
| Vitamin S | E, D & A supplement. Overall composition |
| | to increase the concentration to |
| | super therapeutic levels. |
| Green Tea Oil | Strong Antioxidant & Anti-Inflammatory. |
| | Has pleasant aromic Fragrance. |

### 2. Processes for Making Embodiments of the Topical Composition

A topical composition that incorporates teachings of this disclosure may comprise a complex compounded product manufactured (at least in some embodiments) as follows:

First, the wax that has the highest melting point may be heated first. This may be beeswax, which has a melting point of 62-64° C. (144-147° F.). Beeswax may be melted by heating it to a temperature just above its melting point. Then waxes, butters and other components having progressively decreasing melting points may be added. In some embodiments, the temperature of the composition being manufactured (e.g., the melted beeswax, the mixture, etc.) may be progressively decreased, or cooled. This process may continue until oils are to be added and even, in some embodiments, until all of the components of the topical composition have been added.

Manufacturing a topical composition by adding each component at or near its melting point may prevent the degradation of each component and/or enable the mixture or topical composition to rapidly cool and solidify once all of the components have been mixed and introduced into a mold or cast. In addition, such a process may cause all of the components to be substantially homogeneously dispersed throughout the resulting product, which may prevent lighter, less dense components and heavier, denser components from separating from each other, or layering.

### 3. Working Model in Dispenser

In a further aspect, once a topical composition (e.g., a topical composition that incorporates teachings of this disclosure, etc.) has substantially solidified, it may be assembled with (e.g., introduced into, etc.) a container or dispenser, such as a tube (e.g., a malleable tube, a substantially rigid tube, etc.). A container may be used to contain and dispense the topical composition. Without limitation, a tube that contains or otherwise carries a topical composition may be approximately 5½ inches long and approximately one inch wide. Alternatively, a tube that contains or otherwise carries a topical composition may have a length of approximately 3 inches and a width of approximately ½ inch. Such a tube may comprise a substantially cylindrical member.

In some embodiments, two or more sizes of tubes and, thus, two or more quantities of a topical composition, may be packaged together (e.g., combined in a single sales unit, etc.). The Different quantities of a topical composition may be packaged together for marketing purposes, sold for different uses (e.g., large quantities for application to hands, feet, etc.; small quantities for lips, fingers, etc.).

### 4. Function of the Topical Compositions

A topical composition according to this disclosure may restore damaged, chapped and dried skin and/or lips. The topical composition may be formulated to replace depleted or missing fatty components that are useful for maintaining healthy skin. In one application, a topical composition was applied to damaged, dry and fissured skin every day for a week. The result was a substantial healing and restorative effect on the skin.

### 5. Alternative Embodiments

In one alternative embodiment, a topical composition may be formulated with less beeswax and more shea butter and cocoa butter to change the characteristics of the product for easier use on the lips.

A topical composition may also be specifically formulated for use on hands, feet, heels, elbows and/or knees.

Although the foregoing description sets forth many specifics, these should not be construed as limiting the scope of any of the claims, but merely as providing illustrations of some embodiments and variations of elements or features of the disclosed subject matter. Other embodiments of the disclosed subject matter may be devised which do not depart from the spirit or scope of any of the claims. Features from different embodiments may be employed in combination. Accordingly, the scope of each claim is limited only by its plain language and the legal equivalents thereto.

What is claimed:

1. A topical composition, comprising beeswax, lanolin, shea butter, cocoa butter, castor oil, jojoba oil, avocado oil, flax seed oil, grape seed oil, aloe vera oil, meadowform oil, hemp oil, sea buckthorn, baobab oil, carrot seed oil, arnica oil, vitamin E oil, vitamin D oil, vitamin A oil, tea tree oil, balsam fir oil and green tea oil.

2. The topical composition of claim 1, having proportions substantially equal to a mixture that includes 1400 g of beeswax, 400 g of lanolin, 400 g of shea butter, 400 g of cocoa butter, 200 g of castor oil, 200 g of jojoba oil, 100 g of avocado oil, 100 g of flax seed oil, 50 g of grape seed oil, 50 g of aloe vera oil, 50 g of meadowform oil, 50 g of hemp oil, 50 g of sea buckthorn, 50 g of baobab oil, 25 g of carrot seed oil, 25 g of arnica oil, 50,000 I.U. of vitamin E oil, 50,000 I.U. of vitamin D oil, 50,000 I.U. of vitamin A oil, 15 g of tea tree oil, 5 g of balsam fir oil and 10 g of green tea oil.

3. The topical composition of claim 1, having antiseptic, antiviral, antifungal, anti-bacterial or anti-inflammatory properties.

4. The topical composition of claim 1, having antiseptic, antiviral, antifungal, anti-bacterial and anti-inflammatory properties.

5. The topical composition of claim 1, consisting essentially of the beeswax, the lanolin, the shea butter, the cocoa butter, the castor oil, the jojoba oil, the avocado oil, the flax seed oil, the grape seed oil, the aloe vera oil, the meadowform oil, the hemp oil, the sea buckthorn, the baobab oil, the carrot seed oil, the arnica oil, the vitamin E oil, the vitamin D oil, the vitamin A oil, the tea tree oil, the balsam fir oil and the green tea oil.

6. The topical composition of claim 5, having proportions substantially equal to a mixture that includes 1400 g of beeswax, 400 g of lanolin, 400 g of shea butter, 400 g of cocoa butter, 200 g of castor oil, 200 g of jojoba oil, 100 g of avocado oil, 100 g of flax seed oil, 50 g of grape seed oil, 50 g of aloe vera oil, 50 g of meadowform oil, 50 g of hemp oil, 50 g of sea buckthorn, 50 g of baobab oil, 25 g of carrot seed oil, 25 g of arnica oil, 50,000 I.U. of vitamin E oil, 50,000 I.U. of vitamin D oil, 50,000 I.U. of vitamin A oil, 15 g of tea tree oil, 5 g of balsam fir oil and 10 g of green tea oil.

7. The topical composition of claim 1, consisting of the beeswax, the lanolin, the shea butter, the cocoa butter, the castor oil, the jojoba oil, the avocado oil, the flax seed oil, the grape seed oil, the aloe vera oil, the meadowform oil, the hemp oil, the sea buckthorn, the baobab oil, the carrot seed oil, the arnica oil, the vitamin E oil, the vitamin D oil, the vitamin A oil, the tea tree oil, the balsam fir oil and the green tea oil.

8. A package for a topical composition, comprising:
a package;
at least two dispensers carried by the package, the at least two dispensers having different sizes; and
the topical composition of claim 1 within each of the at least two dispensers.

9. The package of claim 8, wherein one dispenser of the at least two dispensers is labeled for use lips and another dispenser of the at least two dispensers is labeled for use with other body parts.

10. The package of claim 8, wherein the two or more tubes are are packaged in piggy-back fashion.

11. A method for blending a topical composition, comprising:
selecting a plurality of ingredients;
heating an ingredient having a highest melting point of all ingredients of the plurality of ingredients;
adding each other ingredient of the plurality of ingredients in a sequential order based on decreasing melting points and/or decreasing densities.

12. The method of claim 11, further comprising:
gradually cooling a mixture formed by adding each other ingredient in the sequential order.
